(19) United States
(12) Patent Application Publication
(10) Pub. No.: US 2026/0069721 A1
(43) Pub. Date: Mar. 12, 2026

(54) SPRAYABLE NEAR-INFRARED FLUORESCENT CONTRAST AGENT COMPOSITION FOR DUAL-CHANNEL NERVE-TARGETED IMAGING AND USE THEREOF

(71) Applicants: Korea University Research and Business Foundation, Seongbuk-gu (KR); The General Hospital Cooperation d/b/a Massachusetts General Hospital (MGH), Boston, MA (US)

(72) Inventors: Hyun Koo Kim, Seoul (KR); Eunbi Jun, Seoul (KR); Kyungsu Kim, Seoul (KR); Junhee Lee, Seoul (KR); Ok Hwa Jeon, Seoul (KR); Hak Soo Choi, Needham, MA (US); Kai Bao, Harvard, MA (US)

(21) Appl. No.: 19/320,198

(22) Filed: Sep. 5, 2025

Related U.S. Application Data
(60) Provisional application No. 63/691,782, filed on Sep. 6, 2024.

(30) Foreign Application Priority Data
Sep. 4, 2025 (KR) 10-2025-0126133

(51) Int. Cl. A61K 49/00

(52) U.S. Cl. CPC A61K 49/0032

(57) ABSTRACT

Provided are a contrast agent composition capable of specifically staining a nerve tissue, and the like. The composition has the affinity for myelin to specifically stain a nerve tissue compared to other tissues, has a wavelength in a near-infrared (NIR) region of 700 nm, and may stably emit fluorescence in vivo, and thus may be usefully used for real-time fluorescence imaging compared to other tissues.

### CROSS-REFERENCE TO RELATED APPLICATION

This application claims the benefit of U.S. Provisional Application No. 63/691,782, filed on Sep. 6, 2024, and claims priority to Korean Patent Application No. 10-2025-0126133, filed on Sep. 4, 2025, the entire disclosures of which are hereby incorporated by reference.

### TECHNICAL FIELD

The present disclosure relates to a near-infrared fluorescent contrast agent composition for nerve-targeted imaging, use thereof, and the like.

### BACKGROUND ART

Imaging technology using near-infrared fluorescence has great potential in the medical field, particularly in diagnosis and image-guided surgery. In modern medicine, precise imaging of a nerve tissue is a very important task in the diagnosis and treatment of various neurological diseases. In particular, accurate identification and protection of a nerve tissue during surgery are essential to minimize nerve damage and improve the patient's prognosis. In particular, recurrent laryngeal nerve damage occurs in up to 50% of cases after esophagectomy and thyroidectomy, which may cause hoarseness, aspiration, and dyspnea. In addition, cavernous nerve damage has been reported in up to 70% of cases after prostatectomy, which may cause sexual dysfunction and urinary dysfunction. Therefore, accurate identification and preservation of nerves during cancer surgery have become important issues, and to this end, various types of imaging technologies and contrast agents have been developed.

Various methods such as electromyography, ultrasound, and optical coherence tomography have been used to identify nerves during surgery, but these methods have several limitations in wide-ranging imaging and resolution, making it difficult to provide real-time visual feedback, and thus, real-time fluorescent imaging contrast agents using near-infrared wavelengths have been effectively used. Conventional contrast agents have been used to image various biological tissues, including a nerve tissue, by absorbing specific wavelengths in the visible or near-infrared region. However, these contrast agents have low specificity for a nerve tissue, making it difficult to distinguish the nerve tissue from other surrounding tissues, and most conventional contrast agents have specific absorption characteristics for a tumor tissue to have the limitation to simultaneously distinguish a nerve tissue from a tumor tissue.

In particular, nerve damage occurring during cancer surgery, among various surgeries, results in significant morbidity and mortality. Despite advances in surgical techniques and equipment, nerves are currently identified through visible shapes and anatomical locations, which is performed without image guidance during surgery. Branching, trifurcation, medial or anterior displacement, and non-recurrent anatomical variations are also considered risk factors for damage. In order to minimize high morbidity and mortality due to such nerve damage, visual identification of nerves during complex cancer surgery is very important. However, currently available intravenous nerve-targeted compounds do not provide optimal conditions due to high absorption and scattering in tissues by ultraviolet and visible light wavelengths and short residence times in nerves after systemic circulation. In addition, these compounds have inherently high lipophilic property and charges to represent high background binding to adipose tissue, which significantly reduces a signal-to-background ratio (SBR). Relatively high endogenous tissue autofluorescence in the visible light range is also a factor limiting the in vivo use of these fluorophores.

Therefore, the present inventors intend to provide a contrast agent that absorbs a wavelength of 700 nanometers (nm) in a near-infrared (NIR) region, and provide the possibility of clearly distinguishing a tumor tissue from a nerve tissue by using the contrast agent together with a tumor-targeted contrast agent. In particular, if such a contrast agent has high affinity for myelin and may target a nerve tissue more precisely, the present inventors provide the possibility of overcoming the limitations of conventional technologies.

### DISCLOSURE OF THE INVENTION

### Technical Goals

An aspect of the present disclosure is to provide a nerve-targeted contrast agent composition.

Another aspect of the present disclosure is to provide an imaging method including administering the contrast agent composition to a subject.

Yet another aspect of the present disclosure is to provide a dual-channel imaging approach using a sprayable preparation.

However, aspects of the present disclosure are not limited to the aforementioned aspects, and other aspects which are not mentioned may be clearly understood to one of ordinary skill in the art from the following description.

### Technical Solutions

According to an aspect, there is provided a nerve-targeted contrast agent composition including a compound represented by the following Chemical Formula 1 or a pharmaceutically acceptable salt thereof as an active ingredient:

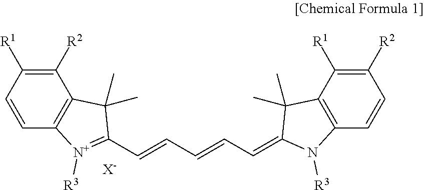

in Chemical Formula 1,
R¹ and R² are the same as or different from each other, and are each independently selected from hydrogen, alkoxy, C₁ to C₆ alkyl, and C₁ to C₆ alkenyl, or combined with each other to form an aromatic ring,
R³ is any one selected from the group consisting of C₁ to C₆ alkyl, C₁ to C₆ alkylsulfonyl, phenylpropyl, and —(CH₂)ₙN⁺(R⁴)₃,
n is an integer of 0 to 4,
R⁴ is C₁ to C₆ alkyl, and
X is a halogen element.

According to an aspect, in Chemical Formula 1, R¹ and R² may be hydrogen, and R³ may be methyl.

According to an aspect, in Chemical Formula 1, X may be I.

According to an aspect, the compound represented by Chemical Formula 1 or the pharmaceutically acceptable salt thereof may specifically bind to myelin.

According to an aspect, the contrast agent may absorb light in a near-infrared (NIR) region having a wavelength of 700 nm to 800 nm. According to an embodiment of the present disclosure, there is provided an imaging method including the following steps:
(1) treating a subject with a contrast agent composition including a compound represented by the following Chemical Formula 1 or a pharmaceutically acceptable salt thereof as an active ingredient; and
(2) detecting a fluorescence signal emitted from the subject.

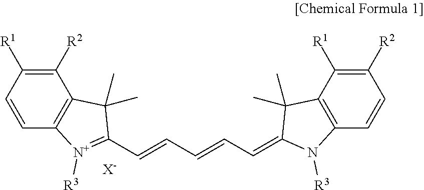

In Chemical Formula 1,
R¹ and R² are the same as or different from each other, and are each independently selected from hydrogen, alkoxy, C₁ to C₆ alkyl, and C₁ to C₆ alkenyl, or combined with each other to form an aromatic ring,
R³ is any one selected from the group consisting of C₁ to C₆ alkyl, C₁ to C₆ alkylsulfonyl, phenylpropyl, and —(CH₂)ₙN⁺(R⁴)₃,
n is an integer of 0 to 4,
R⁴ is C₁ to C₆ alkyl, and
X is a halogen element.

According to an aspect, the imaging method may be performed by further processing a second contrast agent composition having an absorption wavelength of 800 nm or more in addition to the contrast agent composition including the compound represented by Chemical Formula 1 or the pharmaceutically acceptable salt thereof as the active ingredient.

According to an aspect, in the imaging method, the contrast agent composition including the compound represented by Chemical Formula 1 or the pharmaceutically acceptable salt thereof as the active ingredient may specifically stain a nerve tissue, and the second contrast agent composition may specifically stain a tumor tissue.

According to an aspect, in the imaging method, step (1) may be performed by a dyed gauze method.

### Effects of the Invention

According to embodiments of the present disclosure, the nerve-targeted contrast agent has a complex chemical structure including sufficient double bonds to absorb light in a near-infrared (NIR) region of 700 nanometers and emit light of a different wavelength (generally a longer wavelength) from the absorbed light, and is myelin-friendly, so that nerve tissue-specific staining is possible. Through this, the nerve-targeted contrast agent is used together with a second contrast agent that has a different absorption wavelength band in vivo and is specific for tissues other than nerves and thus can be used to distinguish a nerve tissue from other tissues.

The effects of the present disclosure are not limited to the aforementioned effects, and other objects, which are not mentioned above, will be clearly appreciated by one of ordinary skill in the art from the following description.

### BRIEF DESCRIPTION OF THE DRAWINGS

FIG. 1A shows the chemical structure of a nerve-targeted fluorescent contrast agent, and FIG. 1B shows its in vivo imaging.

FIG. 2A shows fluorescence images of the sciatic nerve (SN) and brachial plexus (BP) after staining with NTFP-700 at concentrations of 1 μM to 100 μM, and FIG. 2B shows the quantified signal-to-background ratio (SBR) values (mean±SD).

FIG. 3A shows images of the sciatic nerve (SN) and brachial plexus (BP) after incubation for 1, 3, and 5 minutes, and white arrows indicate nerve bundles. FIG. 3B shows the quantified signal-to-background ratio (SBR) values of the SN and BP (mean±SD).

FIG. 4A shows images of the sciatic nerve (SN) and brachial plexus (BP) after washing for 0 to 10 minutes, and white arrows indicate nerve bundles. FIG. 4B shows the quantified signal-to-background ratio (SBR) values of the SN and BP (mean±SD).

FIG. 5A shows a schematic diagram of the direct administration method, FIG. 5B shows images of the sciatic nerve (SN) obtained by the direct administration method, FIG. 5C shows a schematic diagram of the dyed gauze method, and FIG. 5D shows images of the sciatic nerve (SN) obtained by the dyed gauze method.

FIGS. 6A and 6B show intraoperative dual-channel images of a tumor (cRGD-ZW800-PEG, 800 nm NIR) and nerves (NTFP-700 by direct administration in FIG. 6A and by the dyed gauze method in FIG. 6B, 700 nm NIR) in a mouse tumor model. FIG. 6C shows tumor-to-background and nerve-to-background ratios calculated from quantified fluorescence intensities (mean±SD).

FIG. 7A shows a schematic diagram of dual-channel fluorescence-guided surgery using cRGD-ZW800-PEG and NTFP-700 in a rabbit tumor model. FIG. 7B shows intraoperative dual-channel images of the tumor and nerve, wherein the tumor is indicated in green and the nerve is indicated in blue.

FIG. 8 illustrates histological images of rabbit nerves stained with H&E, NeuroTrace, FluoroMyelin, and NTFP-700 staining.

### DETAILED DESCRIPTION FOR CARRYING OUT THE INVENTION

The present inventors intend to provide a near-infrared fluorescent contrast agent that absorbs a wavelength of 700 nm and has high affinity for myelin, and provide a nerve-targeted imaging method using the same. In addition, a novel imaging method capable of selectively absorbing the contrast agent into a nerve tissue using dyed gauze is also included. These technical features suggest the possibility to be utilized as precise surgical guide and diagnostic tools capable of effectively distinguishing a nerve tissue from a tumor tissue.

This may be achieved by expressing nerve and cancer tissue in two separate near-infrared (NIR) channels (800 nm for cancer and 700 nm for nerve, or vice versa). The superiority of the contrast agent of the present disclosure has the ability to overcome a sprayable formulation, a low signal-to-background ratio (SBR), and tissue autofluorescence, and may provide an improved imaging guidance during surgery compared to current intravenously injectable nerve-targeted compounds.

Therefore, the present disclosure provides a compound represented by the following Chemical Formula 1 or a pharmaceutically acceptable salt thereof.

The present disclosure provides a nerve-targeted contrast agent composition including a compound represented by the following Chemical Formula 1 or a pharmaceutically acceptable salt thereof as an active ingredient:

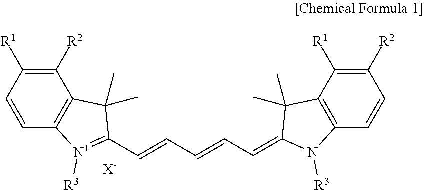

in Chemical Formula 1,
R¹ and R² are the same as or different from each other, and are each independently selected from hydrogen, alkoxy, C₁ to C₆ alkyl, and C₁ to C₆ alkenyl, or combined with each other to form an aromatic ring,
R³ is any one selected from the group consisting of C₁ to C₆ alkyl, C₁ to C₆ alkylsulfonyl, phenylpropyl, and —(CH₂)ₙN⁺(R⁴)₃,
n is an integer of 0 to 4,
R⁴ is C₁ to C₆ alkyl, and
X is a halogen element.

In the present disclosure, the term “alkyl” refers to a group derived from straight-chain or branched-chain saturated aliphatic hydrocarbon having a specified number of carbon atoms and having at least one valence. Examples of such an alkyl group include methyl, ethyl, propyl, isopropyl, butyl, isobutyl, 2-butyl, 3-butyl, pentyl, n-hexyl, and the like, but are not limited thereto.

In the present disclosure, the term “alkenyl” refers to a group having one C═C double bond in the alkyl group. Examples of such an alkenyl group include ethenyl group, propenyl group, butenyl group, and the like, but are not limited thereto.

In the present disclosure, the term “alkylsulfonyl” means that hydrogen at the end of the alkyl group is replaced with —SO₃⁻.

In the present disclosure, the term “halogen” refers to elements belonging to Group 17 of the periodic table, and may be fluorine (F), chloride (Cl), bromine (Br), iodine (I), or the like.

In the present disclosure, the term “alkoxy” refers to an atom group CₙH₂ₙ₊₁O⁻ formed by bonding an oxygen atom to an alkyl group, and examples of such an alkoxy group include methoxy, ethoxy, propoxy, phthaloxy, and the like, but are not limited thereto.

In the present disclosure, the term “pharmaceutically acceptable salt” means a formulation of a compound that does not cause serious irritation to an organism to which the compound is administered and does not impair the biological activity and physical properties of the compound. The pharmaceutically acceptable salt may be obtained by reacting the compound of the present disclosure with inorganic acids such as hydrochloric acid, hydrobromic acid, sulfuric acid, nitric acid, and phosphoric acid; sulfonic acids such as methanesulfonic acid, ethanesulfonic acid, and p-toluenesulfonic acid; and organic carbonic acids such as tartaric acid, formic acid, citric acid, acetic acid, trichloroacetic acid, trifluoroacetic acid, capric acid, isobutanoic acid, malonic acid, succinic acid, phthalic acid, gluconic acid, benzoic acid, lactic acid, fumaric acid, maleic acid, and salicylic acid. In addition, the pharmaceutically acceptable salt may also be obtained by reacting the compound of the present disclosure with bases to form alkali metal salts such as ammonium salt, sodium or potassium salt; salts such as alkaline earth metal salts such as calcium or magnesium salt; salts of organic bases such as dicyclohexylamine, N-methyl-D-glucamine and tris(hydroxymethyl)methylamine; and amino acid salts such as arginine and lysine.

In the present disclosure, “including as the active ingredient” means that the corresponding ingredient is included in an amount required or sufficient to realize a desired biological effect. In actual application, the amount to be included as the active ingredient is determined as an amount for treating a target disease, and may be determined in consideration of factors that do not cause other toxicity. For example, the amount may vary according to various factors such as a disease or condition to be treated, a type of composition to be administered, the size of a subject, the severity of a disease or condition, or the like. Effective amounts of individual compositions may be determined empirically without undue experimentation by those skilled in the art to which the present disclosure pertains.

In the present disclosure, the term “contrast agent” refers to a substance to be administered into the body to strongly and specifically contrast or image cancer cells, etc. in a living body, and is currently widely used in the medical and diagnostic fields to enhance images of tissues and cells. The term of the contrast agent of the present disclosure is not limited to the scope of contrast agents for magnetic resonance imaging (MRI), computed tomography (CT), and positron emission tomography (PET), and is used as the meaning including imaging agents for ultrasound image analysis, imaging agents for fluorescence image analysis, etc.

In addition, the present disclosure provides an imaging method including treating a subject with the contrast agent composition.

In the present disclosure, the term “visualization” is also referred to as imaging, and refers to all methods of visualizing a target subject. In the present disclosure, the imaging may desirably be optical imaging using light.

The “imaging” may be at least one selected from the group consisting of fluorescence, bioluminescence, magnetic resonance imaging (MRI), computed tomography (CT), positron emission tomography (PET), single photon emission computed tomography (SPECT), and combinations thereof, but is not limited thereto.

The imaging in the present disclosure may desirably be dual-channel imaging or dual contrast agent imaging, which may mean simultaneously using two or more contrast agents having different wavelengths that may be distinguished from each other. Desirably, the contrast agent composition of the present disclosure may have a wavelength of 700 nm or more and less than 800 nm, and may specifically stain a nerve tissue, and the second contrast agent composition may have a wavelength of 800 nm or more.

In the present disclosure, the term “subject” or “individual” is not limited to any mammals such as livestock, humans, and the like, which requires imaging by distinguishing a nerve tissue from other tissues, but may be desirably a human.

The contrast agent composition according to the present disclosure may be administered through various routes including oral, transdermal, subcutaneous, intravenous or intramuscular route, and the dose of the active ingredient may be appropriately selected according to various factors such as a route of administration, the age, sex, and weight of a patient, and the severity of a patient. In addition, the composition of the present disclosure may be administered in combination with known compounds capable of increasing the desired effect.

The contrast agent composition according to the present disclosure may desirably be administered by a “dyed gauze method”. The dyed gauze method refers to a method in which commercially available gauze is contained in the contrast agent composition and sufficiently soaked, and then a target nerve tissue requiring imaging is wrapped with the gauze and stained.

Another preferred method of administering the contrast agent composition according to the present disclosure is a spray method. The contrast agent composition according to the present disclosure may be formulated into a formulation suitable for spraying.

The terms used in the embodiments are used for the purpose of description only, and should not be construed to be limited. A singular expression includes a plural expression unless otherwise defined differently in a context. In the present disclosure, it should be understood that term “including” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which embodiments pertain. Terms which are defined in a generally used dictionary should be interpreted to have the same meaning as the meaning in the context of the related art, and are not interpreted as ideal or excessively formal meanings unless otherwise defined in the present disclosure.

In describing the components of the embodiments of the present disclosure, terms including first, second, A, B, (a), (b), and the like may be used. These terms are just intended to distinguish the components from other components, and the terms do not limit the nature, sequence, or order of the components. When it is disclosed that any component is “connected”, “coupled”, or “linked” to the other component, it should be understood that the component may be directly connected or linked to the other component, but another component may be “connected”, “coupled”, or “linked” between the respective components.

Hereinafter, embodiments will be described in detail with reference to the accompanying drawings. However, since various modifications may be made to the embodiments, the scope of the present disclosure is not limited or restricted by these embodiments. It should be understood that all modifications, equivalents and substitutes for embodiments are included in the scope of the present disclosure.

In addition, in the description with reference to the accompanying drawings, like components designate like reference numerals regardless of reference numerals and a duplicated description thereof will be omitted. In describing the embodiments, a detailed description of related known technologies will be omitted if it is determined that they unnecessarily make the gist of the embodiments unclear.

The present disclosure may have various modifications and various embodiments, and specific embodiments will be hereinafter illustrated in the drawings and described in detail in the detailed description. However, the present disclosure is not limited to specific embodiments, and it should be understood that the present disclosure covers all the modifications, equivalents and replacements within the idea and technical scope of the present disclosure. In describing the present disclosure, when it is determined that a detailed description of related known arts may obscure the gist of the present disclosure, the detailed description will be omitted.

### Preparation Example 1. Experimental Method

A sprayable near-infrared fluorescent contrast agent compound for neuroimaging of the present disclosure was synthesized according to the following reaction scheme. Various hydrophobic pentamethine cyanine intermediates were synthesized as follows. 2,3,3-trimethylindolenine (1), 4,5-dimethoxy-2,3,3-trimethyl-3H-indole (2), and 1,1,2-trimethyl-1H-benzo[e]indole (3) reacted with respective alkyl halides in boiling acetonitrile to obtain heterocyclic derivatives (4 to 9). Thereafter, the respective salts (4 to 9) were condensed with a Vilsmeier-Haack reagent to form final pentamethine cyanine. The reaction mixture was purified by open column chromatography or solvent precipitation procedure (using diethyl ether or methyl tert-butyl ether) to obtain analytical purity (at least 95% as measured by LC-ELSD-MS).

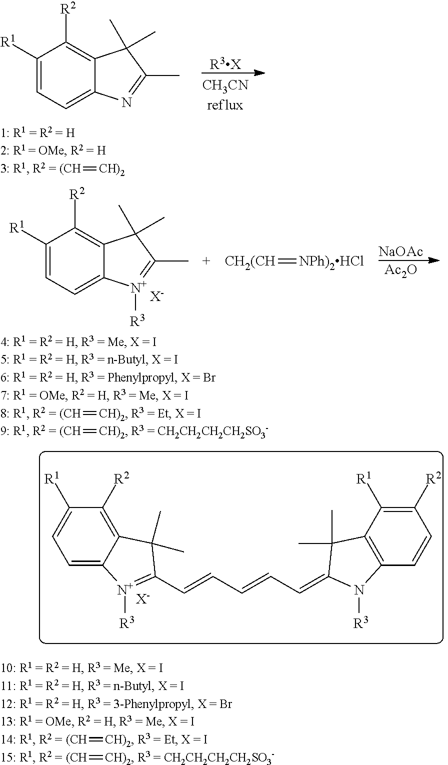

### Preparation Example 1-1. 1,3,3-trimethyl-2-((1E,3E,5E)-5-(1,3,3-trimethylindolin-2-ylidene) penta-1,3-dien-1-yl)-3H-indolium iodide (10 in the Drawing Above, Hereinafter NTFP-700)

Yield 64%; ¹H NMR (400 MHz, MeOD-d₄) δ: 1.71 (s, 12H), 3.63 (s, 6H), 6.28 (d, J=16 Hz, 2H), 6.65 (t, J=12 Hz, 1H), 7.24 (t, J=8 Hz, 2H), 7.29 (d, J=8 Hz, 2H), 7.39 (t, J=8 Hz, 2H), 7.48 (d, J=8 Hz, 2H), 8.25 (t, J=12 Hz, 2H); ¹³C NMR (100 MHz, MeOD-d₄) δ 27.90, 31.71, 50.50, 104.44, 111.83, 123.31, 126.20, 129.71, 142.56, 144.29, 155.52, 175.28. TOF HRMS m/z (M⁺) calculated for [C₂₇H₃₁N₂]⁺ 383.2487, found 383.2474.

### Preparation Example 1-2. 1-butyl-2-((1E,3E,5E)-5-(1-butyl-3,3-dimethylindolin-2-ylidene) penta-1,3-dien-1-yl)-3,3-dimethyl-3H-indolium iodide (11)

Yield: 48%, M.P.>260° C., ¹H NMR (400 MHz, DMSO-d₆) δ: 0.91 (t, J=8 Hz, 6H), 1.37 (q, J=8 Hz, 4H), 1.66 (s, 16H), 4.08 (s, 4H), 6.30 (d, J=16 Hz, 2H), 6.59 (t, J=12 Hz, 1H), 7.23 (s, 2H), 7.38 (s, 4H), 7.60 (d, J=8 Hz, 2H), 8.33 (t, J=12 Hz, 2H). ¹³C NMR (100 MHz, DMSO-d₆) δ: 13.39, 19.11, 26.80, 28.73, 42.84, 48.52, 102.81, 110.74, 122.09, 124.31, 125.25, 128.07, 140.75, 141.65, 153.65, 172.26. TOF HRMS m/z (M⁺) calculated for [C₃₃H₄₃N₂]⁺ 467.7074 found 468.4592.

### Preparation Example 1-3. 2-((1E,3E,5E)-5-(3,3-dimethyl-1-(3-phenylpropyl) indolin-2-ylidene) penta-1,3-dien-1-yl)-3,3-dimethyl-1-(3-phenylpropyl)-3H-indolium iodide (12)

Yield 49%, M.P. 185-187° C., ¹H NMR (400 MHz, DMSO-d₆) δ 1.68 (s, 12H), 2.01 (t, J=8 Hz, 4H), 2.74 (t, J=8 Hz, 4H), 4.15 (t, J=8 Hz, 4H), 6.075 (d, J=12 Hz, 2H), 6.46 (t, J=12 Hz, 1H0, 7.40-7.24 (m, 16H), 7.62 (d, J=8 Hz, 2H), 8.33 (t, J=12 Hz, 2H). ¹³C NMR (100 MHz, DMSO-d₆) δ 27.60, 29.16, 32.58, 43.55, 49.40, 103.58, 111.47, 122.93, 125.19, 125.19, 125.97, 128.71, 128.91, 141.43, 141.43, 142.46, 154.54, 173.11. TOF HRMS m/z (M⁺) calculated for [C₄₃H₄₇N₂]⁺ 591.3734, found 591.2740.

### Preparation Example 1-4. 5-methoxy-2-((1E,3E,5E)-5-(5-methoxy-1,3,3-trimethylindolin-2-ylidene) penta-1,3-dien-1-yl)-1,3,3-trimethyl-3H-indolium iodide (13)

Yield 75%, MP 228-230° C., ¹H NMR (400 MHz, DMSO-d₆): δ 1.66 (s, 12H), 3.56 (s, 3H), 3.81 (s, 6H), 6.165 (d, J=12 Hz, 2H), 6.46 (t, J=12 Hz, 1H), 6.95 (d, J=8 Hz, 2H), 7.30-7.28 (m, 4H), 8.23 (t, J=12 Hz, 2H). ¹³C NMR (100 MHz, DMSO-d₆): δ 26.48, 30.62, 48.45, 55.29, 102.06, 108.43, 111.02, 112.84, 123.73, 135.80, 142.15, 151.94, 156.98, 171.55. TOF HRMS m/z (M⁺) calculated for C₂₉H₃₅N₂O₂ 443.2699 found 443.2692.

### Preparation Example 1-5. 3-ethyl-2-((1E,3E,5E)-5-(3-ethyl-1,1-dimethyl-1H-benzo[e]indole-2 (3H)-ylidene) penta-1,3-dien-1-yl)-1,1-dimethyl-1H-benzo[e]indolium iodide (14)

Yield 79%, MP 266-268° C., ¹H NMR (400 MHz, DMSO-d₆): δ 1.333 (t, J=8 Hz, 6H), 1.962 (s, 12H), 4.297 (t, J=8 Hz, 4H), 6.37 (d, J=12 Hz, 2H), 6.637 (t, J=12 Hz, 1H), 7.51 (t, J=8 Hz, 2H), 6.68 (t, J=8 Hz, 2H), 7.74 (d, J=8 Hz, 2H), 8.08 (t, J=8 Hz, 4H), 8.25 (d, J=8 Hz, 2H), 8.46 (t, J=12 Hz, 2H). ¹³C NMR (100 MHz, DMSO-d₆) δ 11.96, 26.17, 48.04, 50.19, 102.05, 110.90, 121.58, 124.20, 125.07, 127.11, 127.19, 129.39, 129.83, 130.76, 132.72, 138.77, 152.54, 172.71. TOF HRMS m/z (M⁺) calculated for C₃₇H₃₉N₂ 511.3113, found 511.3098.

### Preparation Example 1-6. 4-(2-((1E,3E,5E)-5-(1,1-dimethyl-3-(4-sulfonatobutyl)-1H-benzo[e]indole-2 (3H)-ylidene) penta-1,3-dienyl)-1,1-dimethyl-1H-benzo[e]indolium-3-yl)butane-1-sulfonate (15)

Yield 76%, MP>250° C., ¹H NMR (400 MHz, DMSO-d₆): δ 1.80 (m, J=12 Hz, 9H), 2.00 (s, 12H), 4.2 (q, 4H), 6.4 (d, J=16 Hz, 2H), 6.7 (t, J=12 Hz, 1H), 7.50 (t, J=8 Hz, 2H), 7.70 (t, J=4 Hz, 2H), 8.10 (q, 4H), 8.35 (d, J=8 Hz, 2H), 8.50 (t, J=12 Hz, 2H).

### Experimental Example 1. Experimental Method

### Study Design

This study was designed to simultaneously visualize tumors and nerves during surgery by developing a fluorescent contrast agent NTFP-700, which may be nerve-targeted in a different wavelength channel, 700 nm, used along with a tumor-targeted fluorescent contrast agent cRGD-ZW800-PEG in a wavelength band of 800 nm, which was developed in a previous study, in order to prevent unnecessary nerve damage during surgery by directly administering a nerve-targeted fluorescent agent during cancer surgery.

To optimize the dose, incubation time, and number of washes of a novel nerve-targeted fluorophore, the sciatic nerve (n=4) and the brachial plexus (n=4) of a mouse were utilized. In addition, there is disclosed a dyed gauze method, which may be applied to locations where visualization is difficult due to steep angles caused by cancer or anatomical structures and thus may be easily applied during surgery in actual clinical settings. The effectiveness of a direct administration method and a dyed gauze method according to an angle was evaluated using the sciatic nerve of the mouse.

In addition, mouse (n=4 for each method) and rabbit cancer models (n=4) were utilized to evaluate the applicability during actual cancer surgery. In the previous study, cancer was visualized using an optimized dose of cRGD-ZW800-PEG (50 nmol for mouse, 0.1 mg/kg for rabbit) and nerves were visualized using an optimal dyed gauze method. All fluorescence images of tumor and nerve were simultaneously visualized in real time during tumor surgery using the FIAT-L imaging system (Nawoo Vision).

### Animal Experiment

All surgical procedures, including animal care and handling, were approved by the Institutional Animal Care and Use Committee of Korea University (KOREA-2023-0189). Five-week-old C57BL/6 mice (20 to 25 g) were purchased from Orient Biotech (Seoul, S. Korea), and female New Zealand white rabbits of 2.0 to 2.5 kg were purchased from Koatech (Seoul, S. Korea). All of the mice (five per cage) and rabbits (one per cage) used in each experiment were housed in cages where diets and water were freely provided for 1 to 2 weeks under humane animal care protocols.

A mouse cancer model was established by injecting LLC cells (2×10⁵ cells/ml) into the thigh of mice. In addition, based on a VX2 lung cancer rabbit model method established in the previous study, a VX2 rabbit cancer model was established by injecting a mixture of VX2 carcinoma and Matrigel into the thigh of rabbits. The sciatic nerves of the mice and rabbits were exposed for tumor surgery and imaging.

### Comparison of Neural Specificity of Fluorophores

The neural specificity of NTFP-700 developed in the study was compared using Oxazine 4 (TCI, America), Oxazine 1 (Santa Cruz Biotechnology), and Indocyanine green, which were known to be able to visualize nerves. After exposing the sciatic nerve of mice, 100 μL of each fluorophore was directly applied to the nerve and incubated for 1 minute. After absorbing the fluorophores with gauze and removing the fluorophores, images were acquired using the In vivo Imaging System (Davinch-K, Korea).

### Optimization of Direct Administration Protocol for Fluorophores

To perform a dose optimization study of direct administration of the developed nerve-targeted fluorescent contrast agent, NTFP-700, the sciatic nerve and brachial plexus of mice were exposed (n=4), and for dose optimization, 1 μM 100 μM NTFP-700 was evenly applied to the nerve and the fat and muscle tissue around the nerve. After incubating the NTFP-700 in the nerve area for 1 minute, the contrast agent was absorbed and removed with a clean gauze pad, and then 200 μL of phosphate-buffered saline (PBS) was applied on the nerve and absorbed with gauze, which was repeated 5 times. The optimal incubation time for direct administration was determined by applying 25 μM NTFP-700 to the sciatic nerve and brachial plexus of mice, incubating the NTFP-700 for 1, 3, or 5 minutes, and washing five times with 200 μL of PBS (n=4). The washing was performed by incubating 25 μM NTFP-700 for 1 minute to remove non-specific staining in non-nerve areas, and then images after each wash were collected while washing 1 to 10 times using PBS. Washing was performed by briefly flushing the nerve and tissues around the nerve, and absorbing and removing PBS with clean gauze. The optimization protocol for NTFP-700 by concentration, incubation time, and number of washes was determined based on a nerve-to-background tissue ratio.

### Comparison of Optimized Direct Administration and Dyed Gauze Methods of Fluorophores in Mouse Sciatic Nerve

To compare a direct administration method and a dyed gauze method according to an angle of the nerve, the established optimal conditions of NTFP-700 were used. Direct administration of 100 μL of 25 μM NTFP-700 and a dyed gauze method using soaked fluorophores were used. Two different methods were applied to the nerves located at flat and steep angles according to the anatomical structure to evaluate the usefulness of the methods according to an angle of the nerve location.

### Simultaneous Visualization of Nerve and Tumor Using Dual-Channel Fluorescence Imaging in Mouse Cancer Model

Lewis lung cancer cells were used as a mouse cancer model. An LLC (5×10⁵) cell line was injected into the sciatic nerve area of the mouse, and the cancer model was established after 1 to 2 weeks. A tumor-targeted fluorescent targeting contrast agent cRGD-ZW800-PEG (50 nmol) was administered intravenously 4 hours before surgery, and the sciatic nerve area was exposed. NTFP-700 was applied to the sciatic nerve using the optimized direct administration method and dyed gauze technique, respectively. The nerve (700 nm) and tumor (800 nm) were visualized simultaneously using a near-infrared (NIR) dual fluorescence imaging system.

### Simultaneous Visualization of Nerve and Tumor Using Dual-Channel Fluorescence Imaging in Rabbit Cancer Model

A VX2 rabbit cancer model was developed by referring to a method of forming a cancer model using VX2 in the previous study. Briefly, fresh VX2 tissue was harvested using a surgical blade for tumor transplantation, and washed with phosphate-buffered saline (PBS), and then the surrounding necrotic tumor tissues were removed. Thereafter, the tissue was cut into small pieces and filtered using a 100-μm cell filter to obtain a VX2 suspension. The suspension was centrifuged at 1200 rpm for 3 minutes and then suspended in PBS at a concentration of 1×10⁷ cells/mL. A VX2 carcinoma was mixed with 100 μL Matrigel and prepared using a 1 mL syringe with a 23-gauge needle. A mixture of the VX2 carcinoma and Matrigel was injected into the muscle below the sciatic nerve of rabbits, and a rabbit cancer model was established 2 weeks later.

Similarly, the tumor-targeted fluorescent targeting contrast agent cRGD-ZW800-PEG (50 nmol) was administered intravenously 4 hours before surgery, and the sciatic nerve area was exposed. NTFP-700 was applied to the sciatic nerve using the optimized direct administration method and dyed gauze technique, respectively. The nerve (700 nm) and the tumor (800 nm) were visualized simultaneously using a near-infrared (NIR) dual fluorescence imaging system and the tumor was removed through real-time image-guided surgery.

### Histological Confirmation of Rabbit Sciatic Nerve Tissue

Rabbit sciatic nerve tissues were resected after in vivo nerve staining using an optimized dyed gauze staining technique, mounted in an optimal cutting temperature (OCT) compound, and rapidly frozen in liquid nitrogen. The frozen sample was resected into cryo-slices (10 μm per slice). One slice was stained with H&E and serial slices were used for fluorescence microscopy. Thereafter, the slices were stained with NeuroTrace fluorescent Nissl staining and FluoroMyelin fluorescent myelin staining provided from ThermoFisher Scientific (Waltham, MA), and NTFP-700. Fluorescent and color microscopic images were acquired using an LSM 900 confocal laser scanning microscope (Zeiss, Thornwood, NY) at 2.5× and 10× magnification.

### Statistical Analysis

Statistical analysis was performed using repeated measures or a Tukey's multiple comparison test after using one-way analysis of variance (ANOVA). Statistical significance was set at P<0.05: *P<0.05, **P<0.01, ***P<0.001, and ****P<0.0001. Results were expressed as mean±SD for all image analyses from the Davinch In vivo Imaging System (Davinch-K), FIAT-L Imaging System (Nawoo Vision), and NIR fluorescence microscopy. Statistical analysis and curve fitting were performed using Microsoft Excel and Prism version software (GraphPad).

### Experimental Example 2. Chemical Structure of Contrast Agent and In Vivo Imaging

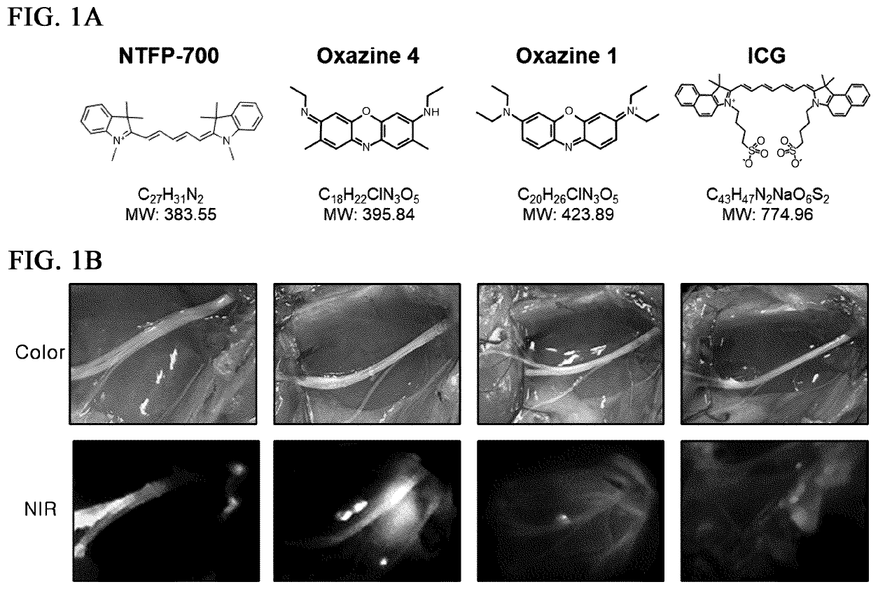

The chemical structures and molecular weights of NTFP-700 which was the nerve-10 targeted contrast agent of the present disclosure, Oxazine 4, Oxazine 1, and Indocyanine Green (ICG) were shown in FIG. 1A. In addition, the fluorescence of NTFP-700, Ox4, Ox1, and ICG directly injected into the mouse sciatic nerve in vivo was shown in FIG. 1B. The NTFP-700, Oxazine 4, and Oxazine 1 were imaged through a 700 nm channel, respectively, and the ICG was imaged through a 800 nm near-infrared (NIR) channel. It was confirmed that NTFP-700, the contrast agent of the present disclosure, was suitable for visualizing the nerve with a wavelength of 700 nm, and was nerve-specifically stained compared to a control group.

### Experimental Example 3. Optimization of NTFP-700 Absorption Dose for Direct Administration in Mice

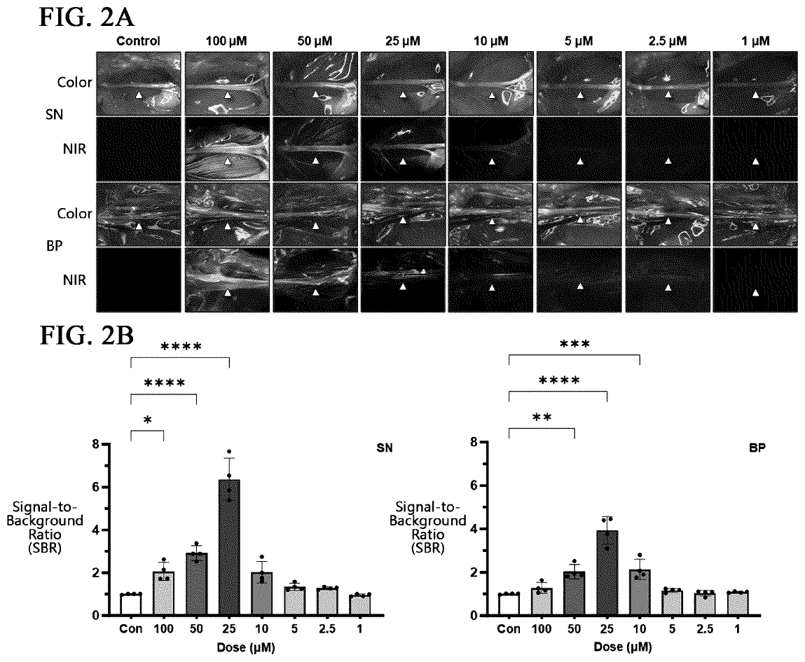

The images of the sciatic nerve (SN) and brachial plexus (BP) after staining at each experimental dose (1 μM to 100 μM) were shown in FIG. 2A. The white arrows indicated the nerve bundles of the mouse. The fluorescence intensities were quantified to calculate the signal-to-background ratio (SBR) of the sciatic nerve (SN) and brachial plexus (BP), which were shown in FIG. 2B (mean±SD). The images were representative images obtained from four independent experiments. ***p<0.0001, ***p<0.001, **p<0.01 and *p<0.05.

As shown in FIG. 2B, the dose of NTFP-700 showing the highest SBR (signal-to-background ratio) was 25 mM, which was confirmed to be the optimal concentration for direct administration.

### Experimental Example 4. Optimization of NTFP-700 Absorption Time for Direct Administration in Mice

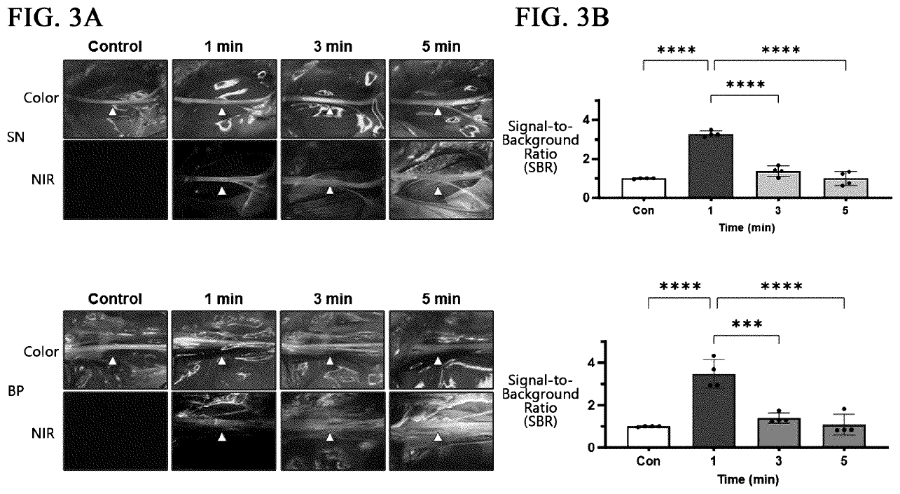

FIG. 3A illustrates images of the sciatic nerve (SN) and brachial plexus (BP) after incubation for each experimental time (1, 3, and 5 minutes), and white arrows indicated nerve bundles in the mouse. The fluorescence intensities were quantified to calculate the signal-to-background ratio (SBR) of the sciatic nerve (SN) and brachial plexus (BP), which were shown in FIG. 3B (mean±SD). The images were representative images obtained from four independent experiments. ****p<0.0001, ***p<0.001.

The optimal incubation time for direct administration of NTFP-700 was 1 minute, which was confirmed to exhibit the highest SBR, and it was shown that the nerve-targeted contrast agent of the present disclosure may be usefully used in situations requiring medical techniques due to a short incubation time.

### Experimental Example 5. Optimization of Washing Protocol of NTFP-700 Absorption for Direct Administration in Mice

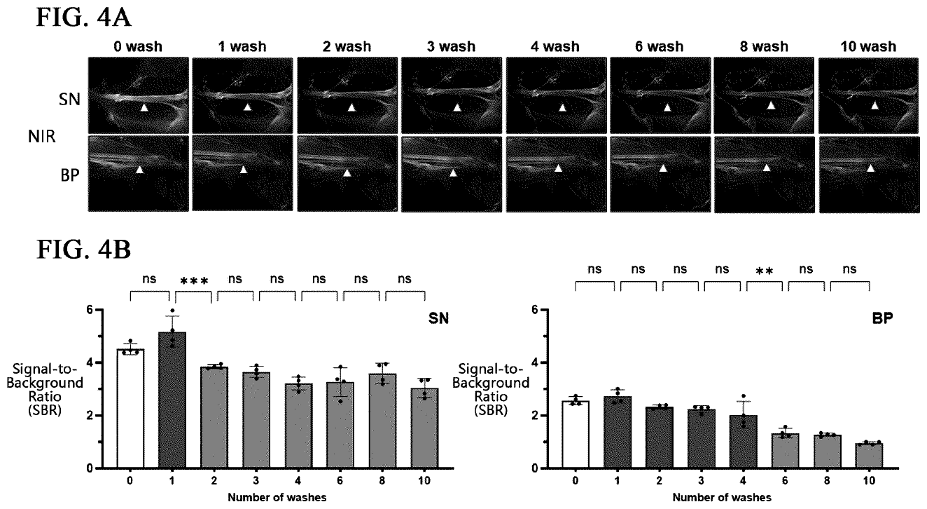

FIG. 4A illustrates images of the sciatic nerve (SN) and brachial plexus (BP) after each washing time (0 to 10 minutes). The white arrows indicated the nerve bundles of the mouse. The fluorescence intensities were quantified to calculate the signal-to-background ratio (SBR) of the sciatic nerve (SN) and brachial plexus (BP), which were shown in FIG. 4B. The images were representative images obtained from four independent experiments. ***p<0.001, **p<0.01.

The optimal number of washes for direct administration of NTFP-700 was 1, which showed the highest SBR. The small number of washes showed that the contrast agent of the present disclosure may be usefully used in situations requiring medical techniques, as described above.

### Experimental Example 6. Comparison of Direct Administration Method and Dyed Gauze Method for Sciatic Nerve Staining

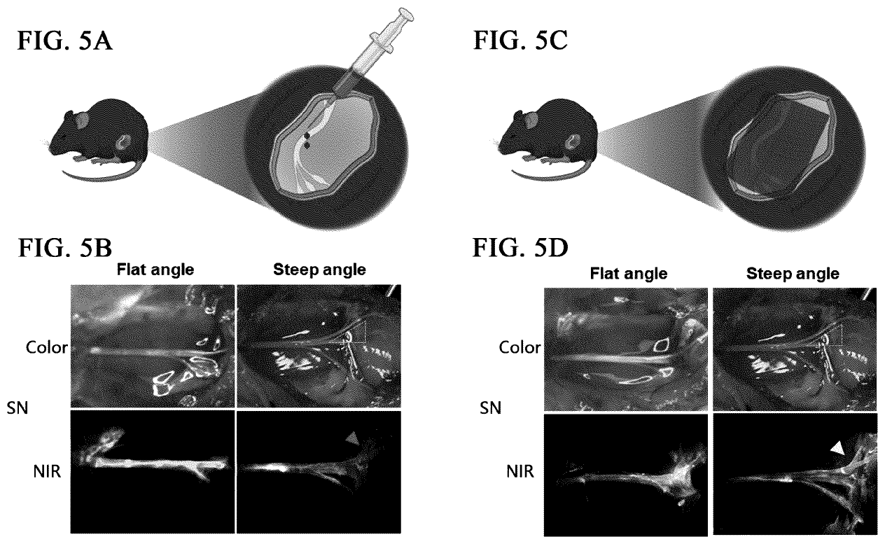

Schematic diagrams of the direct administration method and the dyed gauze method were shown in FIGS. 5A and 5C, respectively. FIGS. 5B and 5D show images of the sciatic nerve (SN) captured at various angles after staining with NTFP-700 using the direct administration method and the dyed gauze method, respectively. The red arrows indicated nerves imaged at steep angles due to the anatomical structures.

Both the direct administration method and the dyed gauze technique were effective in visualizing the nerve at flat angles. However, at the steep angles, the dyed gauze technique was more effective than the direct administration method in that the contrast agent did not flow out.

### Experimental Example 7. Intraoperative Dual-Channel Fluorescence Imaging in Mouse Tumor Model

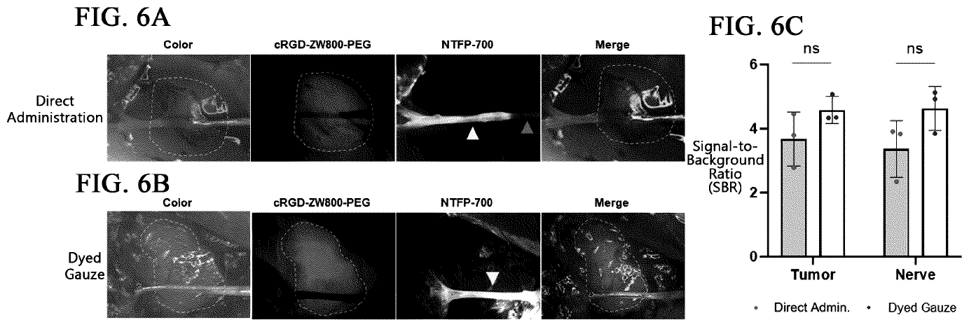

FIGS. 6A and 6B show intraoperative simultaneous dual-channel imaging of tumor (cRGD-ZW800-PEG, 800 nm NIR) and nerves (NTFP-700 direct administration (A) and dyed gauze (B), 700 nm NIR) in a mouse tumor model. The white dotted line indicated the tumor, the red arrow indicated the nerve imaged at a steep angle due to the tumor, and the yellow arrow indicated the nerve imaged at a flat angle. FIG. 6C illustrates the tumor-to-background and nerve-to-background ratios calculated by quantifying the fluorescence intensities (mean±SD). The images were representative images obtained in three independent experiments.

In the dual-channel NIR imaging in the mouse tumor model, both the direct administration method and the dyed gauze technique could be successfully used to simultaneously distinguish the nerves and the tumor using two different colors during cancer surgery.

### Experimental Example 8. Confirmation of Precise Tumor Surgery Using Intraoperative Dual-Channel Fluorescence Imaging Guide in Rabbit Tumor Model

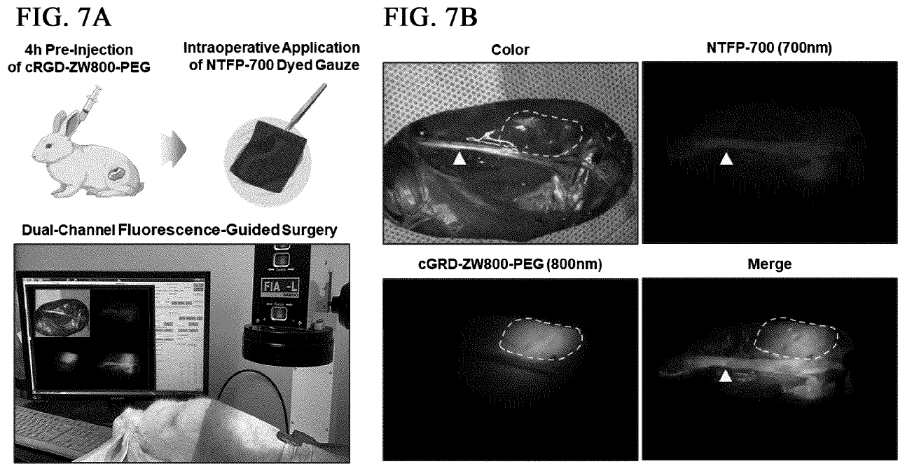

FIG. 7A illustrates a schematic diagram of a dual-channel fluorescence-guided surgery using cRGD-ZW800-PEG and NTFP-700 in a rabbit tumor model. The cRGD-ZW800-PEG was intravenously injected 4 hours before surgery, and the NTFP-700 was administered using the dyed gauze method during surgery. FIG. 7B illustrates representative intraoperative dual-channel imaging results of the tumor (green) and nerve (blue). The white dotted line indicated the tumor, and the yellow arrow indicated the nerve.

In a rabbit subcutaneous tumor model, dual-channel NIR imaging succeeded in simultaneously distinguishing the nerve and the tumor using two different colors during cancer surgery. This method successfully removed the tumor while preserving the nerve.

### Experimental Example 9. Histological Images of Rabbit Nerves Stained with H&E, Neuro Trace, FluoroMyelin, and NTFP-700 Staining

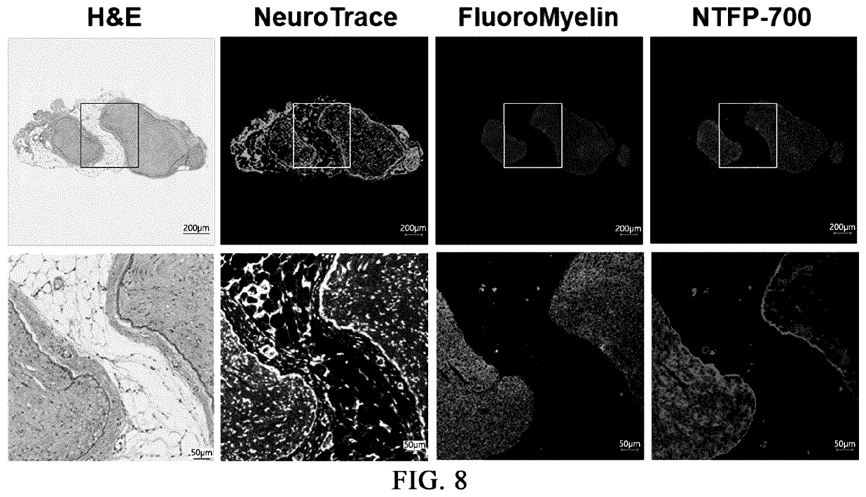

FIG. 8 illustrates representative H&E staining and fluorescence microscopic images of a resected rabbit sciatic nerve. The white square in the first row indicated the location of an image (second row) enlarged 20 times. Scale bars=200 μm and 50 μm. All fluorescence images had the same exposure time and normalization conditions.

The fluorescence signal of NTFP-700 was matched with myelin-specific fluorescence staining, which confirmed the specificity for a nerve tissue. Through this result, it was confirmed that NTFP-700, the nerve-targeted contrast agent of the present disclosure, may target myelin to show nerve tissue-specific staining results.

As described above, although the embodiments have been described by the restricted drawings, various modifications and variations may be applied on the basis of the embodiments by those skilled in the art. For example, even if the described techniques are performed in a different order from the described method, and/or components such as a system, a structure, a device, a circuit, and the like described above are coupled or combined in a different form from the described method, or replaced or substituted by other components or equivalents, an appropriate result may be achieved.

Therefore, other implementations, other embodiments, and equivalents to the appended claims fall within the scope of the claims to be described below.

The present disclosure was performed with the support of the Korea Health Technology Study and Development Program (Project No. RS-2024-00436472) of the Korea Health Industry Development Institute (KHIDI) and was completed with the support of the Ministry of Health and Welfare.

1. A nerve-targeted contrast agent composition comprising a compound represented by the following Chemical Formula 1 or a pharmaceutically acceptable salt thereof as an active ingredient: 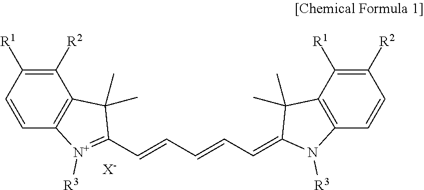
in Chemical Formula 1,
R¹ and R² are the same as or different from each other, and are each independently selected from hydrogen, alkoxy, C₁ to C₆ alkyl, and C₁ to C₆ alkenyl, or combined with each other to form an aromatic ring,
R³ is any one selected from the group consisting of C₁ to C₆ alkyl, C₁ to C₆ alkylsulfonyl, phenylpropyl, and —(CH₂)ₙN⁺(R⁴)₃,
n is an integer of 0 to 4,
R⁴ is C₁ to C₆ alkyl, and
X is a halogen element.

2. The nerve-targeted contrast agent composition of claim 1, wherein in Chemical Formula 1,
R¹ and R² are hydrogen, and
R³ is methyl.

3. The nerve-targeted contrast agent composition of claim 1, wherein in Chemical Formula 1, X is I.

4. The nerve-targeted contrast agent composition of claim 1, wherein the compound represented by Chemical Formula 1 or the pharmaceutically acceptable salt thereof specifically binds to myelin.

5. The nerve-targeted contrast agent composition of claim 1, wherein the contrast agent absorbs light in a near-infrared (NIR) region having a wavelength of 700 nm to 800 nm.

6. An imaging method comprising the following:
(1) treating a subject with a contrast agent composition comprising a compound represented by the following Chemical Formula 1 or a pharmaceutically acceptable salt thereof as an active ingredient; and
(2) detecting a fluorescence signal emitted from the subject. 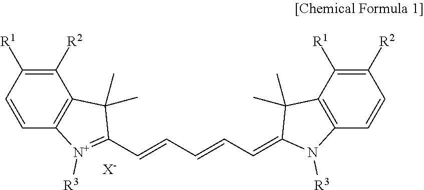
in Chemical Formula 1,
R¹ and R² are the same as or different from each other, and are each independently selected from hydrogen, alkoxy, C₁ to C₆ alkyl, and C₁ to C₆ alkenyl, or combined with each other to form an aromatic ring,
R³ is any one selected from the group consisting of C₁ to C₆ alkyl, C₁ to C₆ alkylsulfonyl, phenylpropyl, and —(CH₂)ₙN⁺(R⁴)₃,
n is an integer of 0 to 4,
R⁴ is C₁ to C₆ alkyl, and
X is a halogen element.

7. The imaging method of claim 6, wherein the imaging method is performed by further processing a second contrast agent composition having an absorption wavelength of 800 nm or more in addition to the contrast agent composition comprising the compound represented by Chemical Formula 1 or the pharmaceutically acceptable salt thereof as the active ingredient.

8. The imaging method of claim 7, wherein in the imaging method, the contrast agent composition comprising the compound represented by Chemical Formula 1 or the pharmaceutically acceptable salt thereof as the active ingredient specifically stains a nerve tissue, and the second contrast agent composition specifically stains a tumor tissue.

9. The imaging method of claim 6, wherein in the imaging method, step (1) is performed by a dyed gauze method or a spraying method.
